(19) United States
(12) Patent Application Publication
(10) Pub. No.: US 2018/0325930 A1
(43) Pub. Date: Nov. 15, 2018

(54) Use of Oligosaccharide Compounds for Activating Angiogenesis

(71) Applicant: Urgo Recherche Innovation et Developpement, Chenove (FR)

(72) Inventors: Marielle Bouschbacher, Chambolle-Musigny (FR); Christelle Laurensou, Dijon (FR)

(73) Assignee: Urgo Recherche Innovation et Developpement, Chenove (FR)

(21) Appl. No.: 15/776,643

(22) PCT Filed: Nov. 15, 2016
(86) PCT No.: PCT/FR2016/052964

§ 371 Date: May 16, 2018

(30) Foreign Application Priority Data
Nov. 17, 2015 (FR) 15 61056

(51) Int. Cl. A61K 31/7024; A61P 17/02

(52) U.S. Cl. CPC A61K 31/7024; A61P 17/02

(57) ABSTRACT

The subject matter of the present invention is a synthetic polysulfated oligosaccharide having 1 to 4 monosaccharaide units, salts thereof, or complexes thereof, at a concentration greater than or equal to 70 mg/mL, for use thereof for activating angiogenesis, in particular for activating angiogenesis during wound healing.

The present invention relates to a synthetic polysulphated oligosaccharide having 1 to 4 monosaccharide units, salts thereof, or complexes thereof, at a concentration greater than or equal to 70 mg/ml, for use thereof for wound healing, in particular for activating angiogenesis during wound healing.

The healing of a wound is a natural biological phenomenon, mammalian tissues being capable of repairing localised lesions by means of repair and regeneration processes specific thereto.

The healing rate and quality of a wound are dependent on the general state of the organism affected, the cause of the wound, the condition and location of the wound, and the onset or not of infection, as well as genetic factors predisposing or not to healing disorders.

The natural healing of a wound takes place essentially according to three successive phases, each of these phases being characterised by specific cellular activities which promote the repair process according to specific chronological sequences: the inflammatory phase, the granulation phase (or proliferative phase), and the maturation phase.

The first phase, the inflammatory phase, commences from the rupture of the blood vessels triggering the formation of a clot (blood coagulation) essentially composed of fibrin and fibronectin, and which will form a temporary matrix. This matrix partially fills the lesion and will enable migration within the damaged area of inflammatory cells recruited to cleanse the wound. The platelets present will also release factors (e.g. cytokine, growth factors) enabling the recruitment of healing cells such as inflammatory cells (neutrophils and macrophages), fibroblasts and endothelial cells.

The second phase corresponds to the development of granulation tissue. Firstly, a colonisation of the wound by fibroblast proliferation is observed. Then, migration of endothelial cells from healthy vessels will enable the formation of new blood vessels (neovascularisation), or angiogenesis, of the damaged tissue. This angiogenesis step is fundamental to initiate healing. In the granulation tissue, the fibroblasts are activated and will be differentiated into myofibroblasts exhibiting significant contractile properties, generated by actin microfilaments, enabling contraction of the wound.

The third phase of the repair process, maturation, is accompanied by restructuring of the granulation tissue. Part of the extracellular matrix is digested by proteases (essentially matrix metalloproteinases (MMP) and elastases), and progressive reorganisation of the extracellular matrix is observed. Progressively, collagen type III, in a majority in the granulation tissue, is replaced by collagen type

I, the primary matrix component of the dermis. At the end of the maturation phase, the fibroblasts, myofibroblasts and vascular cells see the proliferation and/or activity thereof reduced. Then the excess cells die by apoptosis. In parallel with the restructuring of the extracellular matrix and the apoptosis of excess cells, the inflammatory state decreases progressively. This phase is the longest: after approximately one year, the scar is restructured, it is no longer red, or rigid, no longer causes pain and flattens.

Nevertheless, some types of wounds do not heal correctly, the 3 key steps of the process taking place abnormally, despite providing the best possible physicochemical and biological conditions. Indeed, the healing rate and quality of a wound are dependent on intrinsic and extrinsic factors. This repair process may therefore be abnormally prolonged according to:
the cause of the wound;
the condition and location thereof;
the onset of infection caused by the presence of certain infectious agents such as Staphylococcus aureus or Pseudomonas aeruginosa;
the existence of a pre-existing condition (such as diabetes, immune deficiency, venous insufficiency, etc.);
the external environment; or
genetic factors predisposing or not to healing disorders.

These wounds include chronic wounds such as venous ulcers, pressure sores and characteristic wounds of diabetic subjects. Chronic wounds are defined by a lack of healing after a period of 6 weeks from the appearance of the wound, regardless of the treatment applied. In order to treat this type of wound, it may be crucial to speed up the healing process.

The present invention focuses more particularly on the activation of angiogenesis, particularly during wound healing, to enable in particular acceleration of healing.

The invention relates, according to a first aspect, to a synthetic polysulphated oligosaccharide having 1 to 4 monosaccharide units, salts thereof, or complexes thereof, at a concentration greater than or equal to 70 mg/ml, for use thereof for activating angiogenesis, in particular for activating angiogenesis during wound healing.

The invention also relates, according to a second aspect, to a pharmaceutical composition comprising a synthetic polysulphated oligosaccharide having 1 to 4 monosaccharide units, salts thereof, or complexes thereof, at a concentration greater than or equal to 70 mg/ml, for use thereof for activating angiogenesis, in particular for activating angiogenesis during wound healing.

The term “activating angiogenesis” denotes any positive stimulation of revascularisation by means of any action aimed at promoting blood circulation and flow, and more particularly via the increase in the generation or length of capillaries or blood vessels, but also via any modification of the structure of said capillaries or blood vessels on any part of the human or animal body and preferably during wound healing.

### DESCRIPTION OF THE FIGURES

FIG. 1 illustrates the effect of treatment with PBS, VEGF-A or KSOS (potassium sucrose octasulphate salt) on chick embryo chorioallantoic membranes (CAM).

FIGS. 2A, 2B and “C illustrate respectively the parameters of total tube lengths, number of segments and number of branch points of blood vessels, and more particularly the level of induction thereof with respect to a negative control (PBS), observed on chick embryo chorioallantoic membranes (CAM) treated with VEGF-A or KSOS at different concentrations.

### SYNTHETIC POLYSULPHATED OLIGOSACCHARIDES HAVING 1 TO 4 MONOSACCHARIDE UNITS

The oligosaccharides used within the scope of the present invention are synthetic oligomers formed of 1 to 4 monosaccharide units, preferably 1 to 3 monosaccharide units, and more preferentially 1 or 2 monosaccharide units, generally bound together by alpha or beta glycoside bonds. In other words, they consist of mono, di, tri or tetrasaccharides, and preferably mono or disaccharides.

There is no particular limitation in respect of the nature of the monosaccharide units of these polysaccharides. Preferably, they will consist of pentoses or hexoses. By way of example of a monosaccharide, mention may be made of glucose, galactose or mannose. By way of example of a disaccharide, mention may be made of maltose, lactose, sucrose or trehalose. By way of example of a trisaccharide, mention may be made of melezitose. By way of example of a tetrasaccharide, mention may be made of stachyose.

Preferably, the oligosaccharide is a disaccharide, and more preferably sucrose.

The term “polysulphated oligosaccharide” according to the present application denotes an oligosaccharide wherein at least two, and preferably all the hydroxyl groups of each monosaccharide have been substituted by a sulphate group.

Preferably, the polysulphated oligosaccharide used within the scope of the present application is sucrose octasulphate.

The polysulphated oligosaccharides used within the scope of the present invention may be presented in the form of salts or complexes.

By way of example of salts, mention may be made of alkaline metal salts such as sodium, calcium or potassium salts; silver salts; or amino acid salts.

By way of example of complexes, mention may be made of hydroxy aluminium complexes.

Within the scope of the present invention, particularly preferred complexes are the following:
potassium sucrose octasulphate salt;
silver sucrose octasulphate salt; and
hydroxyaluminium sucrose octasulphate complex, commonly known as sucralfate.

In particular, within the scope of the present invention, the polysulphated oligosaccharides used are preferably the potassium salts rather than the aluminium salts of sucrose octasulphate.

The polysulphated oligosaccharides used within the scope of the present invention may be presented in the form of micronised powder or in solubilised form.

An example of polysulphated oligosaccharide used within the scope of the present invention is potassium sucrose octasulphate salt (known as the abbreviation KSOS), marketed in the product Urgotul® Start by URGO Laboratories.

The synthetic polysulphated oligosaccharide according to the invention is used at a concentration greater than or equal to 70 mg/mL, preferably greater than or equal to 100 mg/mL. According to one preferred embodiment, the synthetic polysulphated oligosaccharide according to the invention is used at a concentration between 100 mg/mL and 1000 mg/mL.

### Composition

The invention also relates to a pharmaceutical composition comprising the synthetic polysulphated oligosaccharide described above, at a concentration greater than or equal to 70 mg/mL, for use thereof for activating angiogenesis, in particular for activating the angiogenesis of wounds.

### Additional Active Substance

As a general rule, the oligosaccharide compounds according to the invention may be used alone or in a mixture of two or more with each other, or in combination with one (or a plurality of) other active substance(s).

As a general rule, the active agents are chosen from antibacterials, antiseptics, analgesics, anti-inflammatories, active agents promoting healing, depigmenting agents, antipruritic agents, UV filters, soothing agents, hydrating agents, anti-oxidant agents, and mixtures thereof.

As a general rule, the active agents are chosen from:
antibacterials such as Polymyxin B, penicillins (Amoxycillin), clavulanic acid, tetracyclines, Minocycline, chlorotetracycline, aminoglycosides, Amikacin, Gentamicin, neomycin, silver and salts thereof (Silver sulfadiazine), probiotics, silver salts;
antiseptics such as thiomersal, eosin, chlorhexidine, phenyl mercury borate, hydrogen peroxide, Dakin's antiseptic, triclosan, biguanide, hexamidine, thymol, Lugol, Povidone iodine, Merbromine, Benzalkonium and Benzethonium Chloride, ethanol, isopropanol;
analgesics such as Paracetamol, Codeine, Dextropropoxyphene, Tramadol, Morphine and derivatives thereof, Corticosteroids and derivatives;
anti-inflammatories such as Glucocorticoids, non-steroidal anti-inflammatories, Aspirin, Ibuprofen, Ketoprofen, Flurbiprofen, Diclofenac, Aceclofenac, Ketorolac, Meloxicam, Piroxicam, Tenoxicam, Naproxen, Indomethacin, Naproxcinod, Nimesulide, Celecoxib, Etoricoxib, Parecoxib, Rofecoxib, Valdecoxib, Phenylbutazone, niflumic acid, mefanamic acid;
agents promoting healing such as Retinol, Vitamin A, Vitamin E, N-acetyl-hydroxyproline, Centella Asiatica extracts, papain, silicones, essential oils of thyme, melaleuca, rosemary and sage, hyaluronic acid, Allantoin,—Hema'ltîte (gattefossé), Vitamin C, TEGO Pep 4-17 (evonik), Toniskin (silab), Collageneer (Expanscience), Timecode (Seppic), Gatuline skin repair (gattefossé), Panthenol, PhytoCellTec Alp Rose (Mibelle Biochemistry), Erasyal (libragen), Serilesine (Lipotec), Heterosides of Talapetraka (beyer), Stoechiol (codif), macarose (Sensient), Dermaveil (Ichimru Pharcos), Phycosaccaride AI (Codif);
depigmenting agents such as kojic acid (Kojic Acid SL®—Quimasso (Sino Lion)), Arbutin (Olevatin®—Quimasso (Sino Lion)), the mixture of sodium palmitoylpropyl and white water lily extract (Sepicalm®—Seppic), undecylenoyl phenylalanine (Sepiwhite®—Seppic),
antipruritic agents: hydrocortisone, enoxolone, diphenhydramine, anti-H1 local application antihistamine
hydrating agents such as xpermoist (lipotec), Hyaluronic acid, Urea, fatty acids, Glycerine, Waxes, Exossine (unipex)
UV filters such as Parsol MCX, Parson 1789
soothing agents such as chamomile, bisabolol, xanthalene, glycyrrhebenic acid, tanactine (CPN), Calmiskin (Silab),
antioxidant agents, such as vitamin E.

According to one preferred embodiment, the oligosaccharide compounds according to the invention may be used in combination with an antioxidant agent.

### Galenic Form

The synthetic polysulphated oligosaccharides used within the scope of the present invention may be administered topically, and particularly used in a galenic formulation, such as for example a gel, a solution, an emulsion, a cream, granules or capsules of variable size ranging from a nano or micrometre to a millimetre, which will enable the application thereof directly onto the wound. Alternatively, the compounds used within the scope of the present invention may be used in a solution for subcutaneous injection.

If they are used in a mixture of two or a plurality together or in combination with one or a plurality of other active substances, these compounds may be incorporated in the same galenic formulation or in separate galenic formulations.

Obviously, the quantity of synthetic polysulphated oligosaccharides according to the invention used in the galenic formulation is adapted according to the kinetics sought as well as the specific constraints associated with the nature, solubility, heat resistance thereof, etc.

### Dressing

Preferentially, the synthetic polysulphated oligosaccharide compounds used within the scope of the present invention, or a galenic formulation containing same, will be incorporated in a dressing.

The synthetic polysulphated oligosaccharide compounds, and particularly potassium sucrose octasulphate salt or a galenic formulation containing same may be incorporated in any element of the structure of the dressing provided that this compound can come directly or indirectly in contact with the surface of the wound.

Preferably and in order to promote rapid action, this compound (or a galenic formulation containing same) will be incorporated into the layer of the dressing that comes into contact with the wound or deposited on the surface of the dressing which comes into contact with the wound.

Advantageously, potassium sucrose octasulphate salt (or a galenic formulation containing same) may thereby be deposited, continuously or discontinuously, on the surface intended to come into contact with the wound:
either in liquid form, for example by spraying a solution or suspension containing same;
or in solid form, for example by screening a powder containing same.

The layer or surface coming into contact with the wound may consist for example of an absorbent material such as hydrophilic polyurethane absorbent foam; a textile material such as a compress, such as for example a non-woven material, a film, a web of fibres; an optionally absorbent adhesive material; an optionally adherent interface structure.

As a general rule, the galenic form or the structure of the dressing may be adjusted to obtain a specific rapid or delayed release profile of potassium sucrose octasulphate salt, as needed.

Obviously, the quantity of potassium sucrose octasulphate salt using the galenic formulation or in the dressing will be adapted according to the kinetics sought as well as specific constraints associated with the nature, solubility, heat resistance thereof, etc.

The term dressing is intended to denote, according to the present application, all types of dressings used for treating wounds.

Typically, a dressing comprises at least one layer or matrix, optionally adhesive.

The synthetic polysulphated oligosaccharide compounds according to the invention, or a galenic formulation containing same, may be incorporated in any element of the structure of a dressing, for example in the matrix.

Preferably, and in order to promote rapid action, this compound (or a galenic formulation containing same) may be incorporated into the layer of the dressing that comes into contact with the wound or deposited on the surface of the layer of the dressing which comes into contact with the wound.

Such deposition techniques are well-known to those skilled in the art and some are for example described in the patent application WO 2006/007814.

According to one alternative embodiment of the invention, the synthetic polysulphated oligosaccharide compound according to the invention may be incorporated in an absorbent dressing based on gelling fibres, such as for example the product AQUACEL® marketed by CONVATEC.

Very frequently, when fitting these dressings, nursing staff hold the latter in place using a strip or cover the latter with a secondary element such as a second absorbent dressing or an immobilising strip. It is therefore necessary for the dressing to remain attached to the wound so that nursing staff keep their hands free for positioning these secondary elements. As a general rule, any type of adhesive routinely used in dressings may be used for this purpose.

So as not to damage the healthy tissue or the edges of the wound, particularly when removing the dressing, an adhesive having the property of adhering to the skin without adhering to the wound will be preferred.

By way of example of such an adhesive, mention may thus be made of adhesives based on silicone or polyurethane elastomers, such as silicone or polyurethane gels, and hydrocolloidal adhesives.

Such hydrocolloidal adhesives particularly consist of an elastomeric matrix based on one or a plurality of elastomers chosen from poly(styrene-olefin-styrene) sequenced polymers in association with one or a plurality of compounds chosen from plasticisers, such as mineral oils, tackifying resins and, if required, antioxidants, wherein is incorporated a quantity, preferably small, of hydrocolloids (from 3 to 20% by weight) such as for example sodium carboxymethylcellulose or superabsorbent polymers such as the products marketed under the trade name LUQUASORB® by BASF.

According to one preferred embodiment, the synthetic polysulphated oligosaccharide compounds used within the scope of the present invention, or a galenic formulation containing same, will be incorporated in a dressing comprising a hydrocolloidal adhesive, said polysulphated oligosaccharide being incorporated in said adhesive preferably in a quantity between 1 and 15% by weight, more preferably between 5 and 10% by weight, with respect to the weight of the adhesive.

The formulation of such hydrocolloidal adhesives is well-known to those skilled in the art and described for example in the patent applications FR 2 783 412, FR 2 392 076 and FR 2 495 473.

The use of an adhesive net on the non-woven material makes it possible particularly advantageously to reduce or prevent the risk of small fibrils of textile material coming into contact with the wound and adhering to the tissues, thereby causing a painful sensation on removal, or an obstacle to the wound healing process.

According to a preferred alternative embodiment of the present invention, the synthetic polysulphated oligosaccharide compound according to the invention is incorporated into such an adhesive at a concentration compatible with the solubility thereof and the heat resistance thereof.

On the basis of these criteria, the synthetic polysulphated oligosaccharide compound according to the invention is used preferably in a quantity between 1 and 15% by weight, and more preferably between 5 and 10% by weight, with respect to the total weight of the adhesive.

If it is sought to increase the absorption of this non-woven dressing, the latter may be associated with an additional absorbent layer, and preferably an absorbent layer that does not gel, such as in particularly a compress such as that used in the product URGOTUL® Duo or URGOTUL® Trio, an absorbent hydrophilic foam, preferably a hydrophilic polyurethane foam having an absorption capacity greater than that of the non-woven material such as that used in the product CELLOSORB®.

According to a preferred embodiment, the synthetic polysulphated oligosaccharide compound according to the invention is incorporated in a non-woven dressing, associated with an additional absorbent layer, and preferably an absorbent layer that does not gel, such as in particular a compress.

According to a further embodiment, the synthetic polysulphated oligosaccharide according to the invention is incorporated in a non-woven dressing, associated with an additional absorbent layer, and preferably an absorbent layer that does not gel, such as in particular an absorbent hydrophilic foam, preferably a hydrophilic polyurethane foam having an absorption capacity greater than that of the non-woven material.

The non-woven material and the foam may be associated by means of techniques well-known to those skilled in the art, for example by hot rolling using a thermofusible powder based on TPU polymers/polycaprolactone.

This technique is routinely used for binding non-woven materials intended for the medical market together.

Finally, this foam and the non-woven (when the latter is used alone) may be covered with a substrate to protect the wound from the outside.

This substrate may have a greater size than that of the other layers and rendered adhesive continuously or discontinuously on the face thereof coming into contact with the wound in order to optimise the hold of the dressing during the use thereof, in particular if the wound is situated on non-planar body areas.

This substrate and the adhesive thereof are preferably impermeable to fluids but very permeable to water vapour so as to enable optimal management of the exudates absorbed by the dressing and prevent maceration problems.

Such substrates are well-known to those skilled in the art and consist for example of breathing and impermeable films such as polyurethane films, foam/film or non-woven/film complexes.

### Additive

Besides the active agents, the oligosaccharide compounds according to the invention may be used in combination with one (or a plurality of) additives routinely used in the preparation of dressings. These additives may particularly be chosen from perfumes, preservatives, vitamins, glycerine, citric acid, etc.

The activity of the synthetic polysulphated oligosaccharides according to the invention has been demonstrated in the following non-limiting examples.

### Example: Demonstration of the Effect of Potassium Sucrose Octasulphate Salt (KSOS) on Angiogenesis

In order to demonstrate the effect of potassium sucrose octasulphate (KSOS) on angiogenesis, the CAM model or chicken chorioallantoic membrane model was used. This consists of a vascularised membrane found in bird or reptile eggs. In mammals, this structure forms the placenta. The model for the study will be a chicken ex OVO (without the shell) CAM model.

The experimental conditions are as follows:

### Biological Material

Fertilised chicken eggs were used.

For the study, VEGF-A was used as the positive control.

Potassium sucrose octasulphate salt (KSOS) is presented in the form of a solution at different concentrations, buffered with PBS.

A PBS buffer solution was used as a negative control.

### Experimental Procedure

On receipt, the eggs are stored at 13° C. to stop the development thereof until the time of the study. The eggs are then placed in incubation in a hatcher at 37° C. to enable the development of the embryo.

At 3 days, the eggs are broken unto a weighing cup so as to remove the shell, thereby rendering the embryo and all the structures accessible for future treatment. The ex ovo embryos are then placed back in the hatcher where they will continue the in vitro development thereof.

The embryos will be treated in the vehicle (PBS only), VEGF or different concentration of KSOS.

The treatment area is delimited by a silicone ring 1 cm in diameter and 0.2 in thickness. The treatment volume is 25 μl. The treated embryos are placed back in the hatcher for 48 hours.

After 48 hours of incubation, 10% skimmed milk is injected between the CAM and the yolk to separate the 2 vascularisations and increase the contrast for an optimal photographic shot. A photo is taken of the treatment area with a NIKON AZ 1 OO, NIS-element capture software, with 0.5×4 magnification.

| Group | Treatment | Treatment time |
|---|---|---|
| 1 | PBS | 48 hours |
| 2 | VEGF 0.5 μg (20 μg/mL) | 48 hours |
| 3 | KSOS 3 mg/mL | 48 hours |
| 4 | KSOS 10 mg/mL | 48 hours |
| 5 | KSOS 30 mg/mL | 48 hours |
| 6 | KSOS 50 mg/mL | 48 hours |
| 7 | KSOS 100 mg/mL | 48 hours |

### Results: Photo and Image Processing

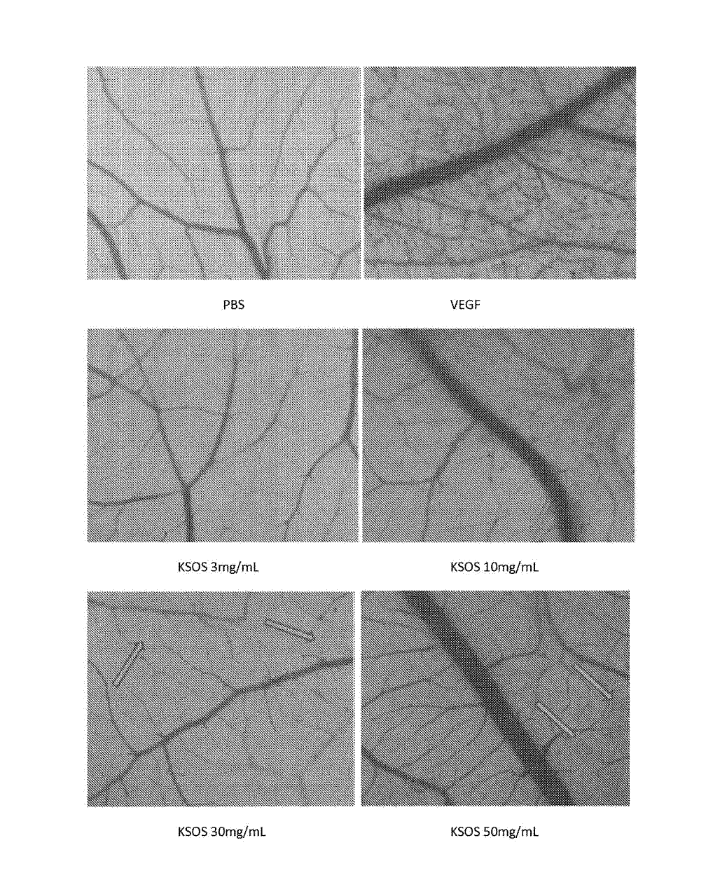

FIG. 1 illustrates the observations of the embryos according to the treatment: the photos are taken at the treatment area.

VEGF induces anarchic neovascularisation of the CAM, as expected.

KOS exhibits a slight vascular change from 30 mg/mL, with respect to the negative control (PBS only) with an accentuation of this effect at higher doses shown on the photos with arrows. Small vessels are found accompanied by a reaction comparable to a hyperaemic reaction (Vasodilation, increase in blood inflow). At 100 mg/mL, a well-developed vascular network is observed, with the appearance of haemorrhagic areas or spots (top arrow). At the 100 mg/mL dose, the results are therefore considered to be conclusive, as they demonstrate angiogenesis activation equivalent to that observed with VEGF-A used as a positive control.

On the basis of these photos, a number of parameters were determined using the image analysis software:
a) the total tube length (sum of the lengths of each vessel in the image),
b) the number of segments (total number of vessels connected to a branch point),
c) the number of branch points (total number of junctions connected to a segment).
b and c are representative of the formation of new characteristic vessels of angiogenesis.

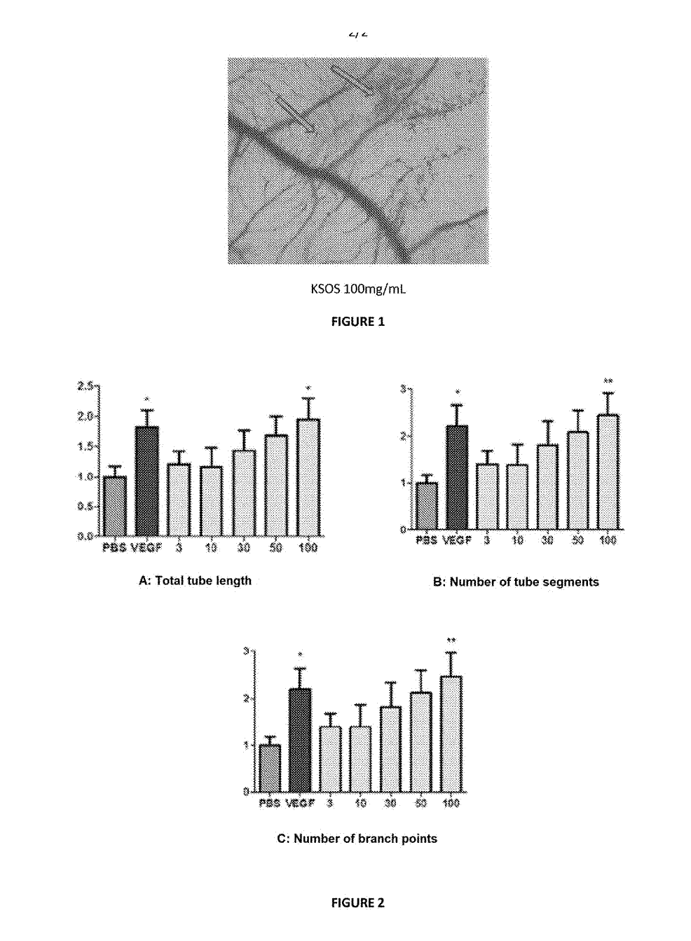

FIG. 2 illustrates these different parameters for each group tested.

As expected, VEGF induces a significant increase in the 3 parameters measured.

KSOS also induces a significant increase in the 3 parameters measured using 100 mg/mL. At lower doses, i.e. at 30 and 50 mg/mL, a poorly developed vascular network is nonetheless visible, but the angiogenesis activation is not considered to be significant with respect to the activation observed for the treatment with VEGF-A or at 100 mg/mL of KSOS.

Therefore, the angiogenesis activation can be reasonably considered to be significant from 70 mg/mL.

KSOS undeniably induces an increase in CAM vascularisation at the treatment area. The development of an increasingly pronounced blood vessel network according to the dose inoculated is clearly quantifiable with the image analysis. The angiogenesis activation demonstrated for KSOS makes it possible to speed up wound healing.

1. A synthetic polysulphated oligosaccharide having 1 to 4 monosaccharide units, salts thereof, or complexes thereof, at a concentration greater than or equal to 70 mg/ml, for use thereof for activating angiogenesis.

2. The oligosaccharide according to claim 1, for use thereof for activating angiogenesis during wound healing.

3. The oligosaccharide according to claim 1, wherein the concentration thereof is greater than or equal to 100 mg/mL.

4. The oligosaccharide according to claim 1, wherein the concentration thereof is between 100 and 1000 mg/mL.

5. The oligosaccharide according to claim 1, wherein the oligosaccharide comprises 1 to 3 monosaccharide units, 1 to 2 monosaccharide units chosen preferably from pentoses and hexoses, as well as the salts and complexes of these compounds.

6. The oligosaccharide according to claim 1, wherein the oligosaccharide chosen from:
potassium sucrose octasulphate salt;
silver sucrose octasulphate salt; and
hydroxyaluminium sucrose octasulphate complex.

7. The oligosaccharide according to claim 1, wherein the oligosaccharide is formulated for administration topically in the form of a composition selected from the group consisting of a gel, a solution, an emulsion, a cream, granule and capsules enabling application directly on the wound.

8. A method of increasing angiogenesis during healing in a subject in need thereof comprising administering the subject the oligosaccharide according to claim 1.

9. A pharmaceutical composition comprising a synthetic polysulphated oligosaccharide having 1 to 4 monosaccharide units, salts thereof, or complexes thereof, at a concentration greater than or equal to 70 mg/mL.
